SEQUENCE LISTING (27 sequences)

SEQ ID NO: 1 - DNA, 17 bp, Artificial Sequence
  Description of Artificial Sequence Synthetic primer
gagcgcggctacagctt

SEQ ID NO: 2 - DNA, 22 bp, Artificial Sequence
  Description of Artificial Sequence Synthetic primer
tccttaatgtcacgcacgattt

SEQ ID NO: 3 - DNA, 18 bp, Artificial Sequence
  Description of Artificial Sequence Synthetic probe
accaccacggccgagcgg

SEQ ID NO: 4 - DNA, 21 bp, Artificial Sequence
  Description of Artificial Sequence Synthetic primer
gggaatttggcgacgtaattc

SEQ ID NO: 5 - DNA, 18 bp, Artificial Sequence
  Description of Artificial Sequence Synthetic primer
gcggaggctgaggaacag

SEQ ID NO: 6 - DNA, 25 bp, Artificial Sequence
  Description of Artificial Sequence Synthetic probe
cacaggtgctctggcccagcacata

SEQ ID NO: 7 - DNA, 20 bp, Artificial Sequence
  Description of Artificial Sequence Synthetic probe
tcacctgggccgcacccttg

SEQ ID NO: 8 - DNA, 24 bp, Artificial Sequence
  Description of Artificial Sequence Synthetic primer
cctgtaccagtccaataccatcct

SEQ ID NO: 9 - DNA, 20 bp, Artificial Sequence
  Description of Artificial Sequence Synthetic primer
tcctgctggtccttcccata

SEQ ID NO: 10 - DNA, 25 bp, Artificial Sequence
  Description of Artificial Sequence Synthetic probe
cacaggtgctctggcccagcacata

SEQ ID NO: 11 - DNA, 21 bp, Artificial Sequence
  Description of Artificial Sequence Synthetic primer
gggaatttggcgacgtaattc

SEQ ID NO: 12 - DNA, 18 bp, Artificial Sequence
  Description of Artificial Sequence Synthetic primer
gcggaggctgaggaacag

SEQ ID NO: 13 - DNA, 25 bp, Artificial Sequence
  Description of Artificial Sequence Synthetic probe
cagccagagatgtgacagccaccgt

SEQ ID NO: 14 - DNA, 17 bp, Artificial Sequence
  Description of Artificial Sequence Synthetic primer
cctgcggacggaatcct

SEQ ID NO: 15 - DNA, 20 bp, Artificial Sequence
  Description of Artificial Sequence Synthetic primer
gctgtgatgagtggcaggct

SEQ ID NO: 16 - DNA, 21 bp, Artificial Sequence
  Description of Artificial Sequence Synthetic probe
tcgccagctacgccctgctca

SEQ ID NO: 17 - DNA, 18 bp, Artificial Sequence
  Description of Artificial Sequence Synthetic primer
gcctcggtgtgcctttca

SEQ ID NO: 18 - DNA, 17 bp, Artificial Sequence
  Description of Artificial Sequence Synthetic primer
cccgtgatgtgcgcaat

SEQ ID NO: 19 - DNA, 29 bp, Artificial Sequence
  Description of Artificial Sequence Synthetic probe
cagtgcctacagtctcgagtctgccagtg

SEQ ID NO: 20 - DNA, 19 bp, Artificial Sequence
  Description of Artificial Sequence Synthetic primer
aggacgcaaggagggtttg

SEQ ID NO: 21 - DNA, 20 bp, Artificial Sequence
  Description of Artificial Sequence Synthetic primer
gtccgccgagtccttactga

SEQ ID NO: 22 - DNA, 28 bp, Artificial Sequence
  Description of Artificial Sequence Synthetic probe
cagccaggatcgctgtctctaacttgca

SEQ ID NO: 23 - DNA, 25 bp, Artificial Sequence
  Description of Artificial Sequence Synthetic primer
actaagcaccctgactatgctatcc

SEQ ID NO: 24 - DNA, 25 bp, Artificial Sequence
  Description of Artificial Sequence Synthetic primer
cttccatcacatcactgaacacttt

SEQ ID NO: 25 - DNA, 22 bp, Artificial Sequence
  Description of Artificial Sequence Synthetic probe
ccgtcatcatgtttggcgtccc

SEQ ID NO: 26 - DNA, 21 bp, Artificial Sequence
  Description of Artificial Sequence Synthetic primer
gtccgagggaatcgactttgt

SEQ ID NO: 27 - DNA, 20 bp, Artificial Sequence
  Description of Artificial Sequence Synthetic primer
gcggctctgtgtgagacgta

(19) United States
(12) Patent Application Publication
(10) Pub. No.: US 2012/0289592 A1
(43) Pub. Date: Nov. 15, 2012

(54) ERCC1 GENE EXPRESSION LEVEL IS ASSOCIATED WITH CLINICAL OUTCOMES IN ESOPHAGEAL CANCER PATIENTS

(75) Inventor: Heinz-Josef Lenz, Altadena, CA (US)

(73) Assignee: University of Southern California

(21) Appl. No.: 13/470,143

(22) Filed: May 11, 2012

Related U.S. Application Data
(60) Provisional application No. 61/486,173, filed on May 13, 2011.

(51) Int. Cl. C12Q 1/68; A61P 35/00; A61K 31/282

(52) U.S. Cl. 514/492; 435/6.11

(57) ABSTRACT

The disclosure provides compositions and methods for identifying a cancer patient, such as an esophageal cancer patient, suitable for a therapy that includes administration of a platinum drug and radiation pre-operatively, based on the expression level of an ERCC1 gene. After determining if a patient is likely to be successfully treated, the disclosure also provides methods for treating the patients.

### CROSS-REFERENCE TO RELATED APPLICATION

This application claims priority under 35 U.S.C. §119(e) to U.S. Provisional Application Ser. No. 61/486,173, filed May 13, 2011, the entire contents of which are incorporated by reference into the present disclosure.

### FIELD OF THE INVENTION

This invention relates to the field of pharmacogenomics and specifically to the application of expression levels of genes to diagnose and treat diseases.

### BACKGROUND

Long-term survival and cure are elusive for the majority of patients with locally advanced esophageal adenocarcinoma. (Jeman, A. et al. (2010) CA Cancer J. Clin. 60:277-300) Although randomized trials conducted in the United States and Europe indicate that neoadjuvant chemotherapy and radiation for esophageal cancer lead to improved survival over surgery alone (Tepper, J. et al. (2008) J. Clin. Oncol. 26:1086-1092; Gaast, A. V. (2010) J. Clin. Oncol. 28:302s, Suppl 15s, Abstr 4004), the failure of radiation therapy and systemic chemotherapy to eradicate all local disease and undetectable foci of metastatic cancer, respectively, is a major impediment to improved survival and cure (von Randen, B. H. et al. (2006) World J. Gastroenterol. 12:6608-6613; Suntharalingam, M. (200) Chest Surg. Clin. N. Am. 10:569-581; Meguid, R. A. et al. (2009) J. Thorac. Cardiovasc. Surg. 138:1309-1317).

Identification of intratumoral molecular parameters predictive for response and overall survival (OS) should enable selection of the optimal therapy for each patient (Ku, G. Y. et al. (2009) Esophageal Cancer: Principles of Practice). Because platinum compounds function by binding to DNA, targeting the nucleotide excision repair pathway to prevent repair of bulky, helix-distorting DNA lesions is attractive. The excision repair cross-complementing 1 (ERCC1) protein is recognized as the rate-limiting enzyme in the nucleotide excision repair pathway, with its gene expression correlating with DNA repair capacity (Vogel, U. et al. (2000) Mutat. Res. 461:197-210). Relatively low intratumoral expression levels of ERCC1 have been associated with improved response and survival for patients with lung, gastric, and colorectal cancer treated with platinum compounds (Metzger, R. et al. (1998) J. Clin. Oncol. 16:309-316; Cobo, M. et al. (2007) J. Clin Oncol. 25:2747-2754). Similarly, low expression levels of thymidylate synthase (TS) have been associated with both response and survival for patients treated with cisplatin and protracted-infusion fluorouracil (PI-FU) (Lenz, H. J. et al. (1997) Prog. Gastric Cancer Res. 2:1295-1300). It remains to be established whether either of these influences response or survival when platinum compounds and fluoropyrimidines are combined with radiation and surgery.

### SUMMARY

Working with the dual hypotheses that pathologic complete response (pCR) rate after neoadjuvant therapy meaningfully affects progression-free survival (PFS) and OS and that mRNA quantitation of genes and genetic polymorphisms of interest predict for pCR and OS in patients with esophageal adenocarcinoma treated with oxaliplatin, PI-FU, radiation, and surgery, This study was designed as a singlearm phase II trial. The goal chosen for pCR rate was 40%.

The disclosure provides compositions and methods for identifying a cancer patient suitable for a therapy that includes administration of a pyrimidine antimetabolite such as 5-fluorouracil. After determining if a patient is sensitive to the treatment and therefore likely to be successfully treated, the disclosure also provides methods for treating the patients.

Thus, in one embodiment, the present disclosure provides a method for aiding in the selection of or selecting or not selecting a cancer patient for a therapy comprising a platinum drug and pre-operative radiation and/or a second course of chemotherapy prior to an operative therapy, comprising determining the intratumoral expression level of an ERCC1 gene in a tumor cell or tumor tissue sample isolated from the patient, wherein the patient is selected for the therapy if the ERCC1 gene expression level is lower than a predetermined value, or the patient is not selected for the therapy if the ERCC1 gene expression level is higher than the predetermined value.

Also provided, in another embodiment, is a method for aiding in the determination of or determining whether or not a cancer patient is suitable for a therapy comprising a platinum drug and pre-operative radiation and/or a second course of chemotherapy prior to an operative therapy, comprising determining the intratumoral expression level of an ERCC1 gene in a tumor cell or tumor tissue sample isolated from the patient, wherein the patient is suitable for the therapy if the ERCC1 gene expression level is lower than a predetermined value, or the patient is not suitable for the therapy if the ERCC1 gene expression level is higher than the predetermined value.

Still further provided, in one embodiment, is a method for aiding in the determination of or determining whether a cancer patient is more likely or less likely to experience progression free survival or overall survival following a therapy comprising a platinum drug and pre-operative radiation and/or a second course of chemotherapy prior to an operative therapy, comprising determining the intratumoral expression level of an ERCC1 gene in a tumor cell or tumor tissue sample isolated from the patient, wherein an ERCC1 gene expression level lower than a predetermined level determines that the patient is more likely to experience progression free survival or overall survival, or an ERCC1 gene expression level lower than the predetermined level determines that the patient is less likely to experience progress free survival or overall survival.

In another embodiment, the present disclosure provides a method for aiding in the treatment of or for treating a cancer patient selected for a therapy comprising an effective amount of a platinum drug and pre-operative radiation based on an intratumoral ERCC1 gene expression level in a tumor cell or a tumor tissue sample isolated from the patient that is lower than a predetermined value, comprising administering to the patient the therapy. In one aspect, the patient was selected by a method comprising determining the intratumoral expression level of the ERCC1 gene in a tumor cell or tumor tissue sample isolated from the patient.

In a further aspect, a method is disclosed for aiding in the treatment of or for treating an esophageal cancer patient selected for a second round of a therapy comprising administration of an effective amount of a platinum drug prior to operative therapy, based on an intratumoral ERCC1 gene expression level in a tumor cell or a tumor tissue sample isolated from the patient that is lower than a predetermined value, wherein the method comprises administering to the patient the therapy.

The platinum drug can be oxaliplatin or equivalents or prodrugs thereof, such as cisplatin. In some aspects, the therapy further comprises a pyrimidine antimetabolite and/or radiation therapy.

In some aspects, the pyrimidine antimetabolite is 5-fluorouracil or an equivalent or prodrug thereof. In yet another aspect, the pyrimidine antimetabolite is 5-fluorouracil or capecitabine. In a particular aspect, the pyrimidine antimetabolite is 5-fluorouracil.

Patients who can benefit from compositions or methods of the present disclosure are those who suffer from at least one cancer of the type of the group metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer. In one aspect, the patient suffers from a gastrointestinal cancer. In another aspect, the gastrointestinal cancer is esophageal cancer. In one aspect, the esophageal cancer is stage II-III esophageal adenocarcinoma.

### BRIEF DESCRIPTION OF THE FIGURES

FIG. 1 is a Kaplan-Meier curve of overall survival in the trial reported in Example 2.

FIG. 2 shows progression-free survival by ERCC1 mRNA levels in 53 patients treated with oxaliplatin-based chemoradiotherapy within the trial reported in Example 2.

FIG. 3 shows overall survival by ERCC1 mRNA levels in 53 patients treated with oxaliplatin-based chemoradiotherapy within the trial reported in Example 3.

### DETAILED DESCRIPTION OF THE DISCLOSURE

Throughout this disclosure, various publications, patents and published patent specifications are referenced by an identifying citation or by an Arabic number within parenthesis, the complete bibliographic citation for which are found immediately preceding the claims. The disclosures of these publications, patents and published patent specifications are hereby incorporated by reference into the present disclosure to more fully describe the state of the art to which this disclosure pertains.

The practice of the present disclosure employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### DEFINITIONS

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “patient” intends an animal, a mammal or yet further a human patient. For the purpose of illustration only, a patient includes but is not limited to a human, a simian, a murine, a bovine, an equine, a porcine, a feline, a canine, or an ovine.

“Gastrointestinal cancer” refers to malignant conditions of the gastrointestinal tract. In one aspect, gastrointestinal cancer includes Gastrointestinal stromal tumors (GIST), esophageal cancer, stomach cancer (also called gastric cancer), liver cancer (also called hepatocellular carcinoma, HCC, or hepatoma), gallbladder cancer, pancreatic cancer, colorectal cancer (e.g., called colon cancer, bowel cancer, and rectal cancer) and anal cancer. In one aspect, gastrointestinal cancer includes esophageal cancer, stomach cancer, liver cancer and colorectal cancer. In another aspect, gastrointestinal cancer includes stomach cancer and colorectal cancer.

“Platinum drugs” refer to any anticancer compound that includes platinum. In an embodiment, the anticancer drug can be selected from cisplatin (cDDP or cis-iamminedichloroplatinum(II)), carboplatin, oxaliplatin, and combinations thereof.

Carboplatin is a chemotherapy drug used against some forms of cancer (mainly ovarian carcinoma, lung, head and neck cancers). It was introduced in the late 1980s and has shown vastly reduced side-effects compared to its parent compound cisplatin. Cisplatin and carboplatin, as well as oxaliplatin or other platinum drugs, are classified as DNA alkylating agents. An equivalent of carboplatin includes, but are not limited to, cisplatin, oxaliplatin and other platinum drugs.

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Pyrimidine antimetabolite includes, without limitation, fluorouracil (5-FU), its equivalents and prodrugs. In one embodiment, a pyrimidine antimetabolite is a chemical that inhibits the use of a pyrimidine. The presence of antimetabolites can have toxic effects on cells, such as halting cell growth and cell division, so these compounds can be used as chemotherapy for cancer.

Fluorouracil (5-FU) belongs to the family of therapy drugs call pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

A therapy comprising a platinum drug includes, without limitation, a pyrimidine antimetabolite alone or alternatively the combination of a pyrimidine antimetabolite with other treatments, that include, but are not limited to, radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20 (5):461-470. An example of such is an effective amount of 5-FU and Leucovorin. Other chemotherapeutics can be added, e.g., oxaliplatin or irinotecan.

The term “adjuvant” cancer patient refers to a patient to which administration of a therapy or chemotherapeutic regimen has been given after removal of a tumor by surgery, usually termed adjuvant chemotherapy. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure.

The phrase “first line” or “second line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “genetic marker” refers to an allelic variant of a polymorphic region of a gene of interest and/or the expression level of a gene of interest.

An “internal control” or “house keeping” gene refers to any constitutively or globally expressed gene. Examples of such genes include, but are not limited to, β-actin, the transferring receptor gene, GAPDH gene or equivalents thereof. In one aspect of the disclosure, the internal control gene is β-actin.

“Overexpression” or “underexpression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

A “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present disclosure, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

Alternatively, a gene expression that is higher than the predetermined value is simply referred to as a “high expression”, or a gene expression that is lower than the predetermined value is simply referred to as a “low expression”.

The phrase “polymorphisms were analyzed” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al. (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

When the expression level of a gene or a genetic marker or polymorphism is used as a basis for selecting a patient for a treatment described herein, the expression level or genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21 (7):1404-1411.

“An effective amount” intends to indicate the amount of a compound or agent administered or delivered to the patient which is most likely to result in the desired response to treatment. The amount is empirically determined by the patient's clinical parameters including, but not limited to the stage of disease, age, gender, histology, sensitivity, toxicity and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival (DFS), time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more desirable clinical outcome as compared to a patient or patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to a patient to patients having the same disease and receiving the same therapy but not possessing the characteristic. As defined herein, the patient is considered suitable for the therapy. In another such aspect, a patient possessing a characteristic may exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, disease free survival, progression free survival or overall survival is weighted more heavily than tumor response in a collective decision making.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“Disease free survival” (DFS) refers to the length of time during and after treatment that the patient remains free of disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

The term “determine” or “determining” is to associate or affiliate a patient closely to a group or population of patients who likely experience the same or a similar clinical response.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

“Having the same cancer” is used when comparing one patient to another or alternatively, one patient population to another patient population. For example, the two patients or patient populations will each have or be suffering from colon cancer.

A “tumor” is an abnormal growth of tissue resulting from uncontrolled, progressive multiplication of cells and serving no physiological function. A “tumor” is also known as a neoplasm.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

### Descriptive Embodiments

The disclosure further provides diagnostic, prognostic and therapeutic methods, which are based, at least in part, on determination of the expression level of a gene of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

Determining whether a subject is suitable or not suitable for cancer treatment of a given type, alternatively, can be expressed as identifying a subject suitable for the cancer treatment or identifying a subject not suitable for the cancer treatment of the given type.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods of this disclosure are useful for the diagnosis, prognosis and treatment of patients suffering from at least one or more cancer of the group: metastatic or non-metastatic rectal cancer, metastatic or non-metastatic colon cancer, metastatic or non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer.

The methods are useful in the assistance of an animal, a mammal or yet further a human patient. For the purpose of illustration only, a patient includes but is not limited to a simian, a murine, a bovine, an equine, a porcine, a feline, a canine, or an ovine.

### Diagnostic Methods

The present disclosure, in one embodiment, provides a method for aiding in the selection of or selecting or not selecting a cancer patient for a therapy comprising a platinum drug, comprising determining the intratumoral expression level of an ERCC1 gene in a tumor cell or tumor tissue sample isolated from the patient, wherein the patient is selected for the therapy if the ERCC1 gene expression level is lower than a predetermined value, or the patient is not selected for the therapy if the ERCC1 gene expression level is higher than the predetermined value.

In one aspect, the patient is selected for the therapy if the ERCC1 gene expression level is lower than a predetermined value. In another aspect, the patient is not selected for the therapy if the ERCC1 gene expression level is higher than the predetermined value.

In one embodiment, provides a method for aiding in the selection of or selecting or not selecting a cancer patient for a therapy comprising a platinum drug, comprising determining the intratumoral expression level of an ERCC1 gene in a tumor cell or tumor tissue sample isolated from the patient, wherein the patient is selected for the therapy if the ERCC1 gene expression level is lower than a predetermined value, or the patient is not selected for the therapy if the ERCC1 gene expression level is higher than the predetermined value. In one aspect, the therapy comprises oxaliplatin. In another aspect, the therapy comprises oxaliplatin and 5-fluorouracil. In another aspect, the therapy further comprises radiation, or in particular external beam radiation. In one aspect, the cancer is esophageal cancer. In another aspect, the cancer is esophageal adenocarcinoma or in particular stage II-III esophageal adenocarcinoma. In one aspect, the patient is selected for the therapy if the ERCC1 gene expression level is lower than the predetermined value. In another aspect, the patient is not selected for the therapy if the ERCC1 gene expression level is higher than the predetermined value.

Also provided, in another embodiment, is a method for aiding in the determination of or determining whether or not a cancer patient is suitable for a therapy comprising a platinum drug, comprising determining the intratumoral expression level of an ERCC1 gene in a tumor cell or tumor tissue sample isolated from the patient, wherein the patient is suitable for the therapy if the ERCC1 gene expression level is lower than a predetermined value, or the patient is not suitable for the therapy if the ERCC1 gene expression level is higher than the predetermined value.

Also provided, in another embodiment, is a method for aiding in the determination of or determining whether or not a cancer patient is suitable for a therapy comprising a platinum drug, comprising determining the intratumoral expression level of an ERCC1 gene in a tumor cell or tumor tissue sample isolated from the patient, wherein the patient is suitable for the therapy if the ERCC1 gene expression level is lower than a predetermined value, or the patient is not suitable for the therapy if the ERCC1 gene expression level is higher than the predetermined value. In one aspect, the therapy comprises oxaliplatin. In another aspect, the therapy comprises oxaliplatin and 5-fluorouracil. In another aspect, the therapy further comprises radiation, or in particular external beam radiation. In one aspect, the cancer is esophageal cancer. In another aspect, the cancer is esophageal adenocarcinoma or in particular stage II-III esophageal adenocarcinoma. In one aspect, the patient is suitable for the therapy if the ERCC1 gene expression level is lower than the predetermined value. In another aspect, the patient is not suitable for the therapy if the ERCC1 gene expression level is higher than the predetermined value.

In one aspect, the patient is suitable for the therapy if the ERCC1 gene expression level is lower than a predetermined value. In another aspect, the patient is not suitable for the therapy if the ERCC1 gene expression level is higher than the predetermined value.

Still further provided, in one embodiment, is a method for aiding in the determination of or determining whether a cancer patient is more likely or less likely to experience progression free survival or overall survival following a therapy comprising a platinum drug, comprising determining the intratumoral expression level of an ERCC1 gene in a tumor cell or tumor tissue sample isolated from the patient, wherein an ERCC1 gene expression level lower than a predetermined level determines that the patient is more likely to experience progression free survival or overall survival, or an ERCC1 gene expression level lower than the predetermined level determines that the patient is less likely to experience progress free survival or overall survival.

In one embodiment, is a method for aiding in the determination of or determining whether a cancer patient is more likely or less likely to experience progression free survival or overall survival following a therapy comprising a platinum drug, comprising determining the intratumoral expression level of an ERCC1 gene in a tumor cell or tumor tissue sample isolated from the patient, wherein an ERCC1 gene expression level lower than a predetermined level determines that the patient is more likely to experience progression free survival or overall survival, or an ERCC1 gene expression level lower than the predetermined level determines that the patient is less likely to experience progress free survival or overall survival. In one aspect, the therapy comprises oxaliplatin. In another aspect, the therapy comprises oxaliplatin and 5-fluorouracil. In another aspect, the therapy further comprises radiation, or in particular external beam radiation. In one aspect, the cancer is esophageal cancer. In another aspect, the cancer is esophageal adenocarcinoma or in particular stage II-III esophageal adenocarcinoma. In one embodiment, an ERCC1 gene expression level lower than a predetermined level determines that the patient is more likely to experience progression free survival or overall survival. In another embodiment, an ERCC1 gene expression level lower than the predetermined level determines that the patient is less likely to experience progress free survival or overall survival.

In one aspect of each of the above embodiments, the cancer patient is suffering from at least one cancer of the type of the group metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer. In another aspect, the cancer patient is suffering from esophageal cancer. In yet a further aspect, the cancer patient is suffering from esophageal adenocarcinoma. The esophageal cancer, in some aspects, is at stage II or III.

The platinum drug can be oxaliplatin or equivalents or prodrugs thereof, such as cisplatin. In some aspects, the therapy further comprises a pyrimidine antimetabolite and/or radiation therapy.

In another aspect, the pyrimidine antimetabolite is 5-fluorouracil or an equivalent or prodrug thereof. In yet another aspect, the pyrimidine antimetabolite is 5-fluorouracil or capecitabine. In a particular aspect, the pyrimidine antimetabolite is 5-fluorouracil.

Methods of determining gene expression levels are known in the art. For the purpose of illustration only, such methods can include determining the amount of a mRNA transcribed from the gene using, for example, a method comprising, or alternatively consisting essentially of, or yet further consisting of, one or more of in situ hybridization, PCR, real-time PCR, or microarray. The methods can be performed on at least one of a fixed tissue, a frozen tissue, a biopsy tissue, a resection tissue, a microdissected tissue, or combinations thereof. Methods of determining protein expression levels are also known in the art, such as, without limitation, immunohistochemistry, ELISA or protein microarrays.

In addition, knowledge of the identity of the expression level of a gene in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

The methods described herein may be performed, for example, by utilizing pre-packaged diagnostic kits, such as those described below, comprising at least one probe or primer nucleic acid described herein, which may be conveniently used, e.g., to determine whether a subject is likely to experience tumor recurrence following therapy as described herein or has or is at risk of developing disease such as colon cancer.

Diagnostic procedures can also be performed in situ directly upon tissue sections (fixed and/or frozen) of primary tissue such as biopsies obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, RAVEN PRESS, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the disclosure also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

This disclosure also provides for a prognostic panel of genetic markers selected from, but not limited to the probes and/or primers to determine gene expression as identified herein. The probes or primers can be attached or supported by a solid phase support such as, but not limited to a gene chip or microarray. The probes or primers can be detectably labeled. In one aspect, provided is a panel of probes and/or primers to determine an intratumoral expression level of ERCC1 in a tumor cell or tumor tissue sample.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the disclosure also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest identified in the experimental section below. Thus, they can be used in the methods of the disclosure to determine which therapy is most likely to treat an individual's cancer.

The methods of the disclosure can use nucleic acids isolated from vertebrates. In one aspect, the vertebrate nucleic acids are mammalian nucleic acids. In a further aspect, the nucleic acids used in the methods of the disclosure are human nucleic acids.

Primers for use in the methods of the disclosure are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the disclosure are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the disclosure will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the disclosure are nucleic acids which are capable of selectively hybridizing to the TS gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the disclosure can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the disclosure may be conjugated to another molecule, e.g., a peptide, hybridization triggered cross-linking agent, transport agent, hybridization-triggered cleavage agent, etc.

The isolated nucleic acids used in the methods of the disclosure can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the disclosure can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Methods of Treatment

This disclosure also provides a method for treating a cancer patient selected for therapy based on the presence of a genotype as described above, comprising, or alternatively consisting essentially of, or yet further consisting of, administering an effective amount of a therapy comprising administration of a platinum drug to the patient, wherein the patient was identified by a method described above, thereby treating the patient.

Thus, in one embodiment, the present disclosure provides a method for aiding in the treatment of or for treating a cancer patient selected for a therapy comprising an effective amount of a platinum drug based on an intratumoral ERCC1 gene expression level in a tumor cell or a tumor tissue sample isolated from the patient that is lower than a predetermined value, comprising administering to the patient the therapy.

In another embodiment, provided is use of a therapy comprising a platinum drug for the manufacture of a medicament for aiding in the treatment of or in treating a cancer patient selected for the therapy based on an intratumoral ERCC1 gene expression level in a tumor cell or a tumor tissue sample isolated from the patient that is lower than a predetermined value.

Another embodiment of the present disclosure provides a therapy comprising a platinum drug for use in aiding in the treatment of or in treating a cancer patient selected for the therapy based on an intratumoral ERCC1 gene expression level in a tumor cell or a tumor tissue sample isolated from the patient that is lower than a predetermined value. In one aspect, the therapy comprises oxaliplatin. In another aspect, the therapy comprises oxaliplatin and 5-fluorouracil. In another aspect, the therapy further comprises radiation, or in particular external beam radiation. In one aspect, the cancer is esophageal cancer.

The disclosure further provides methods for treating patients having solid malignant tissue mass or tumor selected for or identified as being suitable for the treatment. In one aspect, a patient is selected or suitable for the therapy if he or she experiences a relatively longer progression free survival or overall survival than a patient having the same cancer and receiving the same therapy but not identified or determined to be suitable for the therapy.

In one aspect of any of the embodiments, the patient was selected by a method comprising determining the intratumoral expression level of the ERCC1 gene in a tumor cell or tumor tissue sample isolated from the patient.

The platinum drug can be oxaliplatin or equivalents or prodrugs thereof, such as cisplatin. In some aspects, the therapy further comprises a pyrimidine antimetabolite.

In another aspect, the pyrimidine antimetabolite is 5-fluorouracil or an equivalent or prodrug thereof. In yet another aspect, the pyrimidine antimetabolite is 5-fluorouracil or capecitabine. In a particular aspect, the pyrimidine antimetabolite is 5-fluorouracil.

In one aspect, the therapy further comprises radiation therapy. In another aspect, the radiation therapy comprises external beam radiation.

Cancer patients that are suitably treated by these methods include those suffering from at least one cancer of the type of the group: metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, head and neck cancer, ovarian cancer, hormone-refractory prostate cancer, non-metastatic unresectable liver cancer, or metastatic or unresectable locally advanced pancreatic cancer. In one particular aspect, the cancer patient is suffering from esophageal cancer, which can be esophageal adenocarcinoma, such as stage II-III esophageal adenocarcinoma.

To identify the patients suitably treated by the therapy, the genotype of a cell or tissue sample isolated from the patient is determined by assaying any suitable cell or tissue that comprises, or alternatively consists essentially of, or yet further consists of, at least one of a tumor cell, a normal cell adjacent to a tumor, a normal cell corresponding to the tumor tissue type, a blood cell, a peripheral blood lymphocyte, or combinations thereof, which can be in a form of at least one of a fixed tissue, a frozen tissue, a biopsy tissue, a resection tissue, a microdissected tissue, or combinations thereof.

Any suitable method for determining the genotype of the sample can be used in the practice of these methods. For the purpose of illustration only, such methods comprise, or alternatively consist essentially of, or yet further consist of, polymerase chain reaction analysis (PCR), sequencing analysis, restriction enzyme analysis, mismatch cleavage analysis, single strand conformation polymorphism analysis, denaturing gradient gel electrophoresis, selective oligonucleotide hybridization, selective PCR amplification, selective primer extension, oligonucleotide ligation assay, exonuclease-resistant nucleotide analysis, Genetic Bit Analysis, primer-guided nucleotide incorporation analysis PCR, PCR-restriction fragment length polymorphism (PCR-RFLP), direct DNA sequencing, whole genome sequencing, and/or microarray.

The methods are useful to treat patients that include but are not limited to animals, such as mammals which can include simians, ovines, bovines, murines, canines, equines, felines, canines, and humans.

The therapies can be administered by any suitable formulation. Accordingly, a formulation comprising the necessary therapy is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

The chemotherapeutic agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional proviso that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, fluorouracil/oxaliplatin, methotrexate-leucovorin, and the like.

Combinations of chemotherapies and molecular targeted therapies, biologic therapies, and radiation therapies are also well known to the art; including therapies such as trastuzumab plus paclitaxel, alone or in further combination with platinum compounds such as oxaliplatin, for certain breast cancers, and many other such regimens for other cancers; and the “Dublin regimen” 5-fluorouracil IV over 16 hours on days 1-5 and 75 mg/m² cisplatin IV or oxaliplatin over 8 hours on day 7, with repetition at 6 weeks, in combination with 40 Gy radiotherapy in 15 fractions over the first 3 weeks) and the “Michigan regimen” (fluorouracil plus cisplatin or oxaliplatin plus vinblastine plus radiotherapy), both for esophageal cancer, and many other such regimens for other cancers, including colorectal cancer.

In another aspect of the disclosure, the method for treating a patient further comprises, or alternatively consists essentially of, or yet further consists of surgical resection of a metastatic or non-metastatic solid malignant tumor and, in some aspects, in combination with radiation. Methods for treating these tumors as Stage I, Stage II, Stage III, or Stage IV by surgical resection and/or radiation are known to one skilled in the art. Guidelines describing methods for treatment by surgical resection and/or radiation can be found at the National Comprehensive Cancer Network's web site, nccn.org, last accessed on May 27, 2008.

The disclosure provides an article of manufacture, comprising packaging material and at least one vial comprising a solution of the chemotherapy as described herein and/or or at least one antibody or its biological equivalent with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The disclosure further comprises an article of manufacture, comprising packaging material, a first vial comprising the chemotherapy and/or at least one lyophilized antibody or its biological equivalent and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

Chemotherapeutic formulations of the present disclosure can be prepared by a process which comprises mixing at least one antibody or biological equivalent and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject® NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the disclosure locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a therapy or a medicament comprising an effective amount of a chemotherapeutic as described herein for treatment of a human cancer patient having the appropriate expression level of the gene of interest as identified in the experimental examples. Further provided is a therapy comprising a platinum drug, or alternatively a platinum drug therapy, for use in treating a human cancer patient having the appropriate expression level of the gene of interest as identified in the experimental examples.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient and the subject being treated. Single or multiple administrations can be carried out with the dose level and pattern being selected by the treating physician.

### Kits

As set forth herein, the disclosure provides diagnostic methods for determining the gene expression of interest. In some embodiments, the methods use probes or primers or microarrays comprising nucleotide sequences which are complementary to the gene of interest. Accordingly, the disclosure provides kits for performing these methods as well as instructions for carrying out the methods of this disclosure. Thus, in one aspect, this disclosure also provides a kit for use in identifying an adjuvant cancer patient more likely to have tumor recurrence, comprising, or alternatively consisting essentially of, or yet further consisting of, suitable antibodies, primers, probes and/or a microarray for determining an expression level of ERCC1 gene, and instructions for use therein. Examples of suitable primers and probes are provided herein.

In one aspect, the present disclosure provides a kit for use in aiding in the selection of or selecting a cancer patient for a therapy comprising a platinum drug, comprising suitable antibodies, primers or probes or a microarray for determining the intratumoral expression level of ERCC1, and instructions for use therein.

In another aspect, provided is a kit for use in aiding in the determination of or determining whether a cancer patient is suitable for a therapy comprising a platinum drug, comprising suitable antibodies, primers or probes or a microarray for determining the intratumoral expression level of the ERCC1 gene, and instructions for use therein.

Yet another aspect of the present disclosure provides a kit for use in aiding in the determination of or determining whether a cancer patient is likely sensitive to therapy comprising a platinum, comprising suitable primers or probes or a microarray for determining the intratumoral expression level of ERCC1, and instructions for use therein. Sensitivity to a therapy includes, without limitation, whether the patient is likely exhibit a favorable clinical outcome following the treatment, with expect to, for example, disease free survival, progression free survival, overall survival, complete or partial response or toxicity.

The platinum drug can be oxaliplatin or equivalents or prodrugs thereof, such as cisplatin. In some aspects, the therapy further comprises a pyrimidine antimetabolite.

In one aspect of any of the above kits, the pyrimidine antimetabolite is 5-fluorouracil or an equivalent or prodrug thereof. In another aspect, the pyrimidine antimetabolite is 5-fluorouracil or capecitabine. In another aspect, the pyrimidine antimetabolite is 5-fluorouracil.

Briefly and for the purpose of illustration only, one of skill in the art can determine the first and second predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

The components and instructions of the kit are useful for the prognosis and treatment of patients suffering from at least one or more cancer of the group: metastatic or non-metastatic rectal cancer, metastatic or non-metastatic colon cancer, metastatic or non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer, prior to a surgical resection.

Suitable samples for use in the methods of this disclosure include, but are not limited to a fixed tissue, a frozen tissue, a biopsy tissue, a resection tissue, a microdissected tissue, or combinations thereof.

Oligonucleotides “specific for” the gene of interest bind either to the gene of interest or bind adjacent to the gene of interest. For oligonucleotides that are to be used as primers for amplification, primers are adjacent if they are sufficiently close to be used to produce a polynucleotide comprising the gene of interest. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

The kit can comprise at least one probe and/or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the disclosure comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present disclosure. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of tumor cells, or biological fluids such as sputum, blood, serum, plasma, or urine that contain tumor cells or tissues. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest. In some aspects, the kits also include a therapy for treating the identified or selected patient.

As amenable, these suggested kit components may be packaged in a manner customary for use by those of skill in the art. For example, these suggested kit components may be provided in solution or as a liquid dispersion or the like.

### Other Uses for the Nucleic Acids of the Disclosure

The identification of the polymorphic region or the expression level of the gene of interest can also be useful for identifying an individual among other individuals from the same species. For example, DNA sequences can be used as a fingerprint for detection of different individuals within the same species. Thompson, J. S. and Thompson, eds., (1991) GENETICS IN MEDICINE, W B Saunders Co., Philadelphia, Pa. This is useful, e.g., in forensic studies.

The disclosure now being generally described, it will be more readily understood by reference to the following example which is included merely for purposes of illustration of certain aspects and embodiments of the present disclosure, and are not intended to limit the disclosure.

### EXPERIMENTAL

### Example 1

This example tested whether the expression of certain genes is associated with clinical outcome of chemotherapy that includes a platinum drug. The genes included those relating to drug metabolism (DPD, GSTPi, TS and TP) and DNA repair (ERCC1 and XPD). Potential association between clinical outcomes and gene polymorphisms were also tested, which included polymorphisms at GSTP, MTHFR, TS, ERCC1, RAD51, XPD, XRCC1 and XRCC3.

Methods: A total of 92 patients from the trial study (S0356) (oxaliplatin (OXP) plus infusion of 5-fluorouracil (5-FU) and external beam radiation prior to surgery) were eligible for the molecular correlative study. mRNA was extracted from laser-capture-microdissected tumor tissue. After cDNA was prepared by reverse transcription, quantitation of the candidate genes and an internal reference gene (β-actin) was performed using a fluorescence-based real-time detection method (TaqMan). Established gene expression cutoffs were tested (ERCC1<1.7×10⁻³; TS<4.0×10⁻³). DNA was extracted from blood and genotyped using PCR-RFLP. Relevant primer sequences are shown below:

| Gene | Forward Primer (5′-3′) | Reverse Primer (5′-3′) | Taqman Probe (5′-3′) |
|---|---|---|---|
| β-actin | GAGCGCGGCTACAGC | TCCTTAATGTCACGCAC | ACCACCACGGCCGAGCG |
| | TT (SEQ ID NO. 1) | GATTT (SEQ ID NO. 2) | G (SEQ ID NO.3 ) |
| ERCC1 | GGGAATTTGGCGACG | GCGGAGGCTGAGGAAC | CACAGGTGCTCTGGCCCA |
| | TAATTC (SEQ ID NO. 4) | AG (SEQ ID NO. 5) | GCACATA (SEQ ID NO. 6) |

Results: In univariate analysis, ERCC1 gene expression level to be significantly associated with progression-free survival (PFS) and overall survival (OS). Patients with high ERCC1 gene expression levels had worse 2-year PFS (17 vs 67%, p=0.0058) and 2-year OS (37 vs 72%, p=0.047) compared to low gene expression levels. Adjustment for baseline characteristics did not affect the results. ERCC1 gene expression levels were not associated with complete pathologic response (pCR). All the other gene expression levels tested did not show significant association with CO. None of the tested polymorphisms showed any association with clinical outcomes.

Preoperative platinum-based chemoradiation therapy for operable esophageal cancer has improved overall survival (OS) compared to surgery alone. This trial was designed to test oxaliplatin (OXP) plus infusion 5-fluorouracil (5-FU) and external beam radiation prior to surgery for potentially curable EA has produced promising centrally confirmed complete pathologic response (pCR) rate (28.2%). Two-year OS was 55.4%.

The data show that the gene expression level of ERCC1 can predict the clinical outcome of platinum drug treated cancer patients.

### Example 2

In an expansion of the study described in Example 1, chemotherapy- and radiation-naive patients age 18 years or older with histologically documented esophageal adenocarcinomas starting at least 26 cm from the incisors and invading no more than 2 cm into gastric cardia were eligible for this study. A Zubrod performance status of 0 to 2 was required. Tumors had to be clinical stage II or III according to the American Joint Commission on Cancer staging system (sixth edition). It was assumed that patients with a mass in the esophagus visualized by either computed tomography (CT) or magnetic resonance imaging (MRI) had at least a primary T2 tumor. Patients without an esophageal mass or enlarged lymph nodes documented by CT scan or MRI were required to undergo endoscopic ultrasonography (EUS) to demonstrate primary tumor invasion into the muscularis propria (stage IIA). A positron emission tomography (PET) scan was required before eligibility confirmation. Thus, staging was performed noninvasively by CT or MRI, PET scan, and, in selected cases, EUS. Before registration, all patients were to be evaluated by their medical oncologist, radiation oncologist, and surgeon.

Within 28 days of registration, patients were required to have granulocytes≧1,500/mL, platelets≧100,000/mL, and hemoglobin≧10.0 g/dL. Eligibility required serum creatinine≦1.5 X institutional limits of normal, and serum albumin and direct bilirubin had to be within the institutional limits of normal. Patients with peripheral neuropathy≧grade 2 and/or patients with any inflammatory process in the lungs were ineligible.

Within 14 days of registration, one hematoxylin and eosin slide, 10 unstained slides, 20 formalin-fixed paraffin-embedded (FFPE) tumor tissue sections 10 μm thick of the patient's esophageal tumor biopsy (alternatively, a paraffin block could be substituted), and one purple-top tube of blood for genetic polymorphism analysis were to be sent to the Southwest Oncology Group (SWOG) Tumor Bank to be stored at −80° C. After esophagectomy, similar tissue specimens were sent for central pathology review. This study was conducted adhering to the Reporting Recommendations for Tumor Marker Prognostic Studies as applicable. All trial patients signed an informed consent to the clinical protocol and consented to giving an adequate pretreatment blood sample for genotyping and for permission to use their paraffin embedded tumor tissue to quantitate the genes of interest.

Genotyping and mRNA Quantification

Details about DNA extraction from blood and single nucleotide polymorphism genotyping are provided in the Appendix (online only). The genes, reference identification numbers, location, function, primers, and restriction enzymes are listed in Table A1. All gene expression levels were measured at a Clinical Laboratory Improvement Amendments—approved laboratory, Response Genetics (Los Angeles, Calif.); microdissection was performed on all FFPE tumor samples to ensure that only tumor cells were dissected. Details about microdissection, mRNA isolation technique, and the procedure for quantitation of genes of interest are summarized below.

Microdissection mRNA Isolation, and Quantification by Real-Time Polymerase Chain Reaction

Manual microdissection using a light microscope was performed on all tumor samples to ensure that more than 80% tumor cells were dissected. RNA isolation from paraffin-embedded samples was performed according to a proprietary procedure defined by Response Genetics (Los Angeles, Calif.; U.S. Pat. No. 6,248,535). After RNA isolation, cDNA was prepared from each sample as described previously (Chomczynski, P. et al. (1987) Anal. Biochem. 162:156-159; Lord, R. V. et al. (2000) J. Gastrointest. Surg. 4:135-142). Quantitation of genes of interest and an internal reference (β-actin) cDNA was performed using a fluorescence-based real-time detection method (ABI PRISM 7900HT Sequence Detection System [TaqMan]; Applied Biosystems, Foster City, Calif.) as previously described (Gibson U E, Heid C A, Williams P M. Genome. Res. 1996; 6:995-1001). The polymerase chain reaction mixture consisted of 1,200 nmol/L of each primer; a 200 nmol/L probe; 0.4 U of AmpliTaq Gold Polymerase; 200 nmol/L of dATP, dCTP, dGTP, and dTTP; 3.5 mmol/L of MgCl₂; and 1× TaqMan Buffer A containing a reference dye added to a final volume of 20 μL (all reagents from Applied Biosystems). Cycling conditions were 50° C. for 2 minutes and 95° C. for 10 minutes followed by 46 cycles at 95° C. for 15 seconds and 60° C. for 1 minute. All samples were amplified in triplicate. For each sample, parallel TaqMan polymerase chain reactions were performed for each gene of interest and the β-actin reference gene to normalize for input cDNA. The obtained ratio between the values provides relative gene expression levels for the gene locus investigated.

TABLE A1
Primers and Probes Used for mRNA Quantification

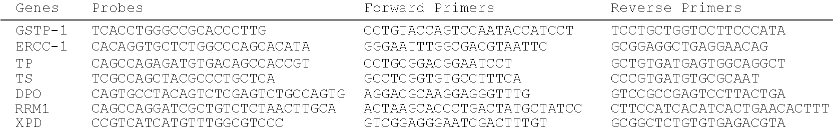

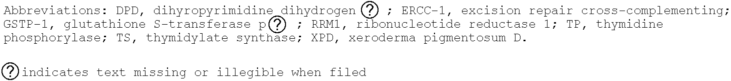

### Treatment

Treatment consisted of preoperative chemotherapy and radiation, surgery, and postoperative chemotherapy. Oxaliplatin 85 mg/m² as a 2-hour intravenous infusion was given on days 1, 15, and 29. On day 8, PI-FU was administered through a central venous catheter at 180 mg/m² over 24 hours continuously through day 43. Dose escalations were not permitted. Dose reductions for clinical toxicities (peripheral neuropathy, nausea/vomiting, diarrhea, oral mucositis, esophagitis, and skin and cardiac toxicities) were outlined in the protocol and specified for each agent or, when appropriate, for both. Guidelines for dose reductions for hematologic toxicities were outlined for each agent. Patients requiring more than two dose reductions were taken off the trial.

External-beam radiation therapy (EBRT) was initiated on day 8, concurrent with start of PI-FU. Intensity-modulated radiation therapy was not allowed. EBRT with megavoltage linear accelerators (≧6 MV) delivered radiation to multiple fields. Patients were treated 5 days a week at 1.8 Gy/d for 25 fractions to a total dose of 45 Gy. At least two fields were treated daily; if a three-field technique was used, all three fields were treated daily. The target volume was defined as the primary tumor and regional lymph node basin that would ordinarily be resected at surgery. If the primary tumor was in the distal third of the esophagus, the target volume was enlarged to include celiac lymph nodes. All clinically involved lymph nodes were given at least a 2-cm margin. Dosage was prescribed at the isocenter. No modifications in radiation treatment were allowed.

After completion of preoperative radiation and chemotherapy, restaging scans and endoscopic evaluation were repeated. Responding or stable patients were operated on 4 to 6 weeks after completion of radiation and chemotherapy. Patients who experienced progression to inoperability for cure were removed from study but were observed for survival.

Acceptable surgical approaches to esophagectomy included transhiatal and transthoracic routes, with either a high intrathoracic or cervical anastomosis. Gastrectomy with esophagojejunostomy was not permitted. An extensive thoracic lymphadenectomy was encouraged, including subcarinal (station 7), paraesophageal (station 8), and inferior pulmonary ligament (station 9) lymph nodes. In the abdomen, the lymphadenectomy included all parahiatal and upper paragastric lymph nodes, extending to the base of the left gastric pedicle (stations 15 to 17). A resection was considered potentially curative (R0) when all visible tumor was removed with negative margins.

Postoperative chemotherapy commenced between 4 and 10 weeks after surgery. Postoperative chemotherapy consisted of oxaliplatin 85 mg/m² on days 1, 15, and 29 and PI-FU 180 mg/m² over 24 hours on days 1 through 29.

### Definition of Response

Pathologic postoperative stage was the only response recorded. A pCR was defined by the pathologist if no cancer could be identified in the resected esophagus and lymph nodes (ypT0N0M0). Staging was not final until a single pathologist central review was conducted. PFS was measured from time of study registration to the time of documented disease progression, symptomatic global deterioration, or death from any cause. OS was measured from the date of registration to the date of death from any cause.

### Statistical Considerations

The primary goal of this study was to evaluate pCR probability (based on surgery and central pathology review) for patients with potentially curable esophageal adenocarcinoma. The study was designed to test the null hypothesis that this combination therapy would result in a true pCR probability of 25% or less against the alternative hypothesis of a true pCR probability of 40% or greater. A two-stage design was used. After the first 45 patients were enrolled, the pCR rate was to be evaluated. If 11 or more patients achieved a pCR, the trial would be allowed to accrue to completion. If 29 of 85 eligible patients achieved a pCR, this would be considered evidence that this regimen would be of interest for future study, regardless of the translational outcome. All treated and eligible patients were considered in the pCR rate. The trial was designed to have power of 89% when the true pCR probability is 40% and a significance level of P=0.04.

The correlative hypothesis for 50356 was that tumor expressions less than 1.7 for ERCC1 and less than 4.0 for TS would be associated with pCR, PFS, and OS. For other genes, median expression levels were explored as the threshold between low and high expression. For each of the polymorphisms included in this analysis, dominant, recessive, codominant, and additive models were explored. No P value adjustments for multiple comparisons were made.

Because of sample size limitations, the primary analyses consisted of univariate models. Multivariate models adjusting for age and disease site (esophageal v gastroesophageal junction) were also explored. For all analyses, results from models adjusting for baseline factors were nearly identical to those from the corresponding univariate models.

Recursive partitioning models were fit to the expression level data to explore whether the categorization of patients into biomarker-based risk groups could be optimized (Breiman, L. et al. (1984) Classification and Regression Trees. Belmont, C A, Wadsworth International Group), and a permutation-based resampling method (Hilsenbeck, S. et al. (1996) Stat. Med. 15:103-112) was used to adjust the P values associated with outcome differences between subgroups identified by this method. The part library for statistical software was used for this portion of the analysis (Therneau, T. M. et al. (http://cran.r-project.org/web/packages/rpart/index.html)).

In accordance with SWOG procedure, toxicity and accrual monitoring for this study were the responsibility of the study coordinator, study statisticians, and the SWOG Disease Committee Chair. Adverse events and toxicities were monitored on an ongoing basis, with formal reports every 6 months.

### Results

Between Feb. 5, 2005, and Aug. 1, 2008, a total of 98 patients were registered. Interim analysis confirmed the requisite pCR rate and safety profile to complete accrual. One patient withdrew consent before therapy could be administered; four patients were ineligible on the basis of histology (n=1) and issues of timing for biopsies and scans (n=3). Demographics and clinical characteristics are listed in Table 1 for the 93 patients evaluable for response, survival, and toxicity. The evaluable patients included 87 men (93.5%) and six women. Race was reported for 90 patients, of whom 86 (95.5%) were white. Primary disease site was the esophagus in 55 patients (60.4%), gastroesophageal junction in 36 patients (39.6%), and not classified in two patients. Preclinical staging by CT scans, EUS, and/or PET scan defined 54 patients (58.1%) as having stage III and 39 patients (41.9%) as having stage 11 disease. With the exception of one patient whose data are missing, all eligible patients had a performance status of 0 (59.0%) or 1 (41.0%).

TABLE 1
Baseline Patient Demographics and Clinical Characteristics

| | No. of Patients | |
|---|---|---|
| Characteristic | (N = 93) | % |
| Age, years | | |

| | |
|---|---|
| Median | 62.2 |
| Range | 41.6-83.1 |

| | | |
|---|---|---|
| Sex | | |
| Male | 87 | 94 |
| Female | 6 | 6 |
| Race | | |
| White | 86 | 96 |
| Other | 4 | 4 |
| Missing | 3 | |
| Performance status | | |
| 0 | 54 | 59 |
| 1 | 38 | 41 |
| Missing | 1 | |
| Primary site | | |
| Esophagus | 55 | 60 |
| Gastroesophageal junction | 36 | 40 |
| Missing | 2 | |
| Disease stage | | |
| II | 39 | 42 |
| III | 54 | 58 |

TABLE 2
Maximum Grade of Adverse Events Experienced by Patients

| | Grade 3 | | Grade 4 | | Grade 5 | |
|---|---|---|---|---|---|---|
| | No. of | | No. of | | No. of | |
| Toxicity | Patients | % | Patients | % | Patients | % |
| Blood/bone marrow | 9 | 9.7 | 7 | 7.5 | 0 | 0 |
| Constitutional | 24 | 25.8 | 0 | 0 | 0 | 0 |
| (fatigue/anorexia) | | | | | | |
| GI (diarrhea/nausea/ | 37 | 39.8 | 1 | 1.1 | 0 | 0 |
| mucositis) | | | | | | |
| Infection | 9 | 9.7 | 9 | 3.2 | 0 | 0 |
| Metabolic (hypokalemia | 10 | 10.8 | 2 | 2.2 | 0 | 0 |
| hyponatremia/renal) | | | | | | |
| Neurologic | 2 | 2.2 | 1* | 1.1 | 0 | 0 |
| Pulmonary | 11 | 11.8 | 6 | 6.4 | 2† | 2.2 |

*One patient had a cerebrovascular accident.
†Two patients had acute respiratory distress syndrome.

Seventy-nine patients (84.9%) underwent esophagectomy. Eight patients had either R1 or R2 resections; the operative data on eight patients are not available; 63 patients underwent an RO resection. Two patients (2.2%) died postoperatively. Thirty-six patients (38.7%) completed postoperative therapy.

Forty-four patients (47.3%) and 18 patients (19.4%) had grade 3 or grade 4 treatment-related toxicities, respectively. The most common grade 3 toxicities were GI in 37 patients (39.8%) and flu-like symptoms in 24 patients (25.8%). The most common grade 4 toxicities were hematologic (7.5%) and pulmonary (6.5%). Two patients (2.2%) died of pulmonary infections before surgery. Table 2 lists the most common toxicities reported in this trial.

TABLE 3
Univariate Analysis of the Association Between mRNA Expression and the Number of Patients
Having Specific Gene Quantitation With Median Expressions Levels, OS, PFS, and pCR

| | OS | | | PFS | | | pCR | | | No. of | Expression Level | |
|---|---|---|---|---|---|---|---|---|---|---|---|---|
| Genes | HR | 95% CI | P | HR | 95% CI | P | OR | 95% CI | P | Patients | Median | Range |
| GSTP-1 | 0.75 | 0.38 to 1.46 | .39 | 1.02 | 0.54 to 1.92 | .98 | 1.40 | 0.41 to 4.76 | .58 | 55 | 1.61 | 0.52-8.73 |
| ERCC-1 | 2.72 | 1.21 to 6.10 | .015 | 2.77 | 1.32 to 5.90 | .0070 | 0.93 | 0.27 to 3.19 | .91 | 53 | 1.92 | 0.33-5.29 |
| TP | 0.90 | 0.45 to 1.91 | .77 | 0.78 | 0.40 to 1.51 | .46 | 1.48 | 0.43 to 6.10 | .83 | 52 | 0.93 | 1.36- [symbol] 8.45 |
| TS | 0.91 | 0.40 to 1.66 | .56 | 0.84 | 0.42 to 1.85 | .60 | 1.01 | 0.28 to 3.58 | .99 | 50 | 3.32 | 0.92-8.82 |
| DPD | 0.72 | 0.33 to 1.60 | .42 | 0.85 | 0.31 to 1.37 | .28 | 0.02 | 0.48 to 8.49 | .82 | 39 | 0.57 | 0-2.32 |
| RRM1 | 1.06 | 0.41 to 2.75 | .91 | 1.15 | 0.46 to 2.86 | .78 | 1.00 | 0.19 to 5.29 | 1.00 | 26 | 1.75 | 0.35-5.91 |
| XPD | 0.41 | 0.11 to 1.53 | .18 | 0.46 | 0.14 to 1.49 | .19 | 2.33 | 0.31 to 17.55 | .41 | 19 | 1.88 | 0.76-3.60 |

NOTE:
HRs are for the comparison between high and low expression.
For ERCC-1 and TS, the thresholds between high and low expression were 1.7 and 4.0, respectively, based on prior results.
For all other genes, the median expression level was used as the threshold value.
Abbreviations:
DPD, dihydropyrimidine dehydrogenase,
SRCC-1, excision repair cross-complementing;
SSTP-1, glutatione S-transferase pi 1;
HR, hazard ratio;
OR, odds ratio;
OS, overall survival;
pCR, pathologic complete response;
PFS, progression-free survival;
RRM1, ribnucleotide reductase 1;
TP, thymidine phosphorytase;
TS, thymidylate synthase;
XPD, xefoderma pigmantosum D. [symbol]
indicates data missing or illegible when filed

Twenty-seven patients (29.0%) had pathology reports stating no cancer could be found in the resected specimen. Central review of these specimens found residual disease in one patient. Hence, 26 patients (28.0%; 95% CI, 19.1% to 38.2%) had central pathology confirmation of T0N0 esophageal lesions after treatment. Among all resected patients, 22 (27.8%) had cancer in lymph nodes (stage III).

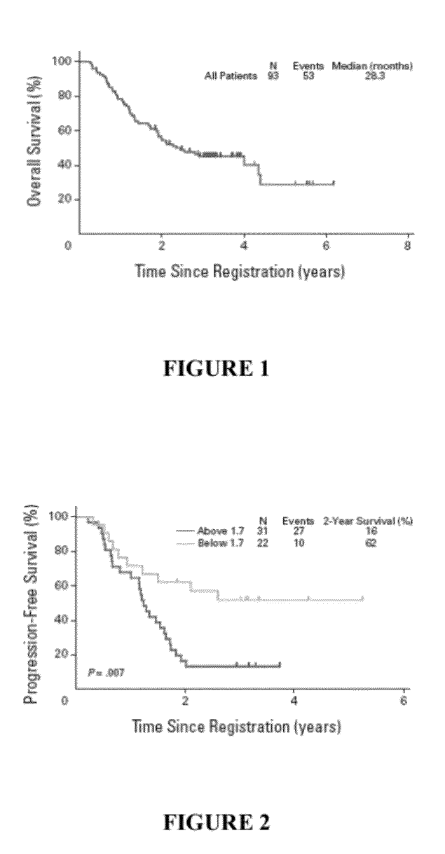

With a median follow-up time of 39.2 months, 53 patients (57.0%) have died. The current Kaplan-Meier estimates of median and 3-year OS are 28.3 months (95% CI, 22.4 to 52.4 months) and 45.1% (95% CI, 34.8% to 55.4%), respectively. Current estimates of median and 3-year PFS are 19.7 months (95% CI, 14.7 to 28.3 months) and 36.8% (95% CI, 26.9% to 46.6%), respectively. The Kaplan-Meier plot for OS is shown in FIG. 1. Patients achieving pCR had a statistically significant survival advantage over patients who did not (P=0.01).

Germline DNA was extracted for analysis from all 91 available blood samples. Of the 91 FFPE tissue samples submitted, sufficient tumor tissue was obtained to perform gene expression of at least one gene in 55 patients. Outcomes and demographic characteristics of patients evaluable for gene expression analysis (n=55) were not significantly different from those of the study population as a whole.

The median ERCC1 mRNA expression in relation to the β-actin gene was 1.92 (range, 0.33 to 5.29), and median TS mRNA expression in relation to the β-actin gene was 3.32 (range, 0.92 to 8.62). Twenty two (41.5%) of 53 patients had ERCC1 mRNA levels below the predefined cutoff of 1.7. Twenty-seven (54.0%) of 50 patients had TS mRNA levels below the predefined cutoff of 4.0. Gene expression data are listed in Table 3. Using the established cutoff values for ERCC1 and TS, no significant association with baseline characteristics was found, including pre-therapy stage.

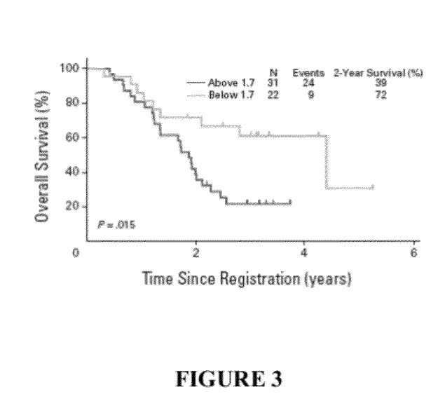

Patients with an ERCC1 gene expression level greater than 1.7 had significantly worse PFS (hazard ratio [HR], 2.77; 95% CI, 1.32 to 5.80; P=0.0070) and OS(HR, 2.72; 95% CI, 1.21 to 6.10; P=0.015) than patients with ERCC1 mRNA expression levels≦1.7. Kaplan-Meier plots of PFS and OS by ERCC1 expression are shown in FIGS. 2 and 3, respectively. ERCC1 gene expression did not influence pCR. The predefined TS gene expression cutoff of 4.0 was not associated with survival or pCR. None of the other tested genes were associated with survival or pCR, using the median mRNA expression levels as cutoff points (Table 3).

A recursive partitioning analysis explored whether optimization of expression level cut point selection might yield improved marker based risk group identification. Only PFS was analyzed with this method, because there were inadequate events for the OS analysis and too few responders for the pCR analysis. Among all genes listed in Table 3, only ERCC1 entered the recursive-partitioning model. The optimal split for ERCC1 mRNA expression using recursive partitioning was 1.66 (adjusted P=0.032).

In the univariate analysis, nominally significant associations were observed between OS and the following two polymorphisms: TS 5-UTR (2R/3R v 2R/2R; HR, 2.39; 95% CI, 0.98 to 5.80; P=0.055) and XPD Lys751Gln (AC v AA; HR, 1.84; 95% CI, 1.00 to 3.36; P=0.049). However, comparison of homozygous patients for these polymorphisms did not yield a significant result.

### Discussion

The hypothesis behind this trial was that pCR rate derived from neoadjuvant therapy of the esophagus drives PFS and OS. The failure to hit the target of 40% for pCR and the resulting PFS and OS suggest the hypothesis was correct, because the pCR rate of 28.0% did not lead to a striking advantage in either PFS or OS over recent single-arm trials for patients with esophageal adenocarcinoma. Indeed, the pCR rate observed in this trial was not high enough to reject the null hypothesis. Nevertheless, this is an active preoperative regimen that can be administered safely without increasing surgical morbidity and mortality. The fact that less than 40% of our patients could be treated postoperatively suggests that consideration be given to a second course of chemotherapy before surgery so that all systemic therapy is completed before surgery.

In perspective, the pCR rate and the median survival time of 28.3 months reported here are similar to recent single-arm trial results reported for patients with esophageal adenocarcinoma in North America and Europe (Urba, S. G. et al. (2001) J. Clin. Oncol. 19:305-313; Walsh, T. N. et al. (1996) N. Engl. J. Med. 335:462-467; Burmeister, B. H. et al. (2005) Lancet Oncol. 6:659-668; Stahl, M. et al. (2009) J. Clin. Oncol. 27:851-856). The Cancer and Leukemia Group B (CALGB) 9781 trial, which was terminated early, is a remarkable exception and justified the goal of investing the S0356 trial with a goal of 40% pCR. The CALGB study reported a pCR rate of 40% and a 4.5-year median survival. Unfortunately, CALGB 9781 enrolled only 30 patients in the treatment arm (Tepper, J. et al. (2008) J. Clin. Oncol. 26:1086-1092). A recent report of a phase III randomized trial from the Netherlands is also noteworthy because it reports a median survival time of approximately 4 years for patients with esophageal cancer treated preoperatively with carboplatin, paclitaxel, radiation, and surgery. However, the inclusion of squamous cell esophageal tumors may have influenced these results. (Gaast, A. V. et al. (2010) J. Clin. Oncol. 28:302s, Suppl 15s, Abstr 4004)

This data demonstrate that patients with ERCC1 mRNA levels greater than the predefined cutoff of 1.7 had significantly worse PFS and OS when compared with patients with levels below this cutoff, including dramatic differences in terms of 2-year PFS (39% v 72%, respectively) and 2-year OS (16% v 62%, respectively). These results provide strong arguments that ERCC1 mRNA expression is associated with PFS and OS in locally advanced esophageal adenocarcinoma treated with this regimen. To the best of our knowledge, this is the first report demonstrating in a prospective fashion that ERCC1 mRNA expression is associated with survival in patients with adenocarcinoma of the esophagus or gastroesophageal junction treated with a potentially curable trimodality strategy. ERCC1 mRNA expression was not associated with pCR. It is possible that the few patients with pCR and evaluable ERCC-1 mRNA quantitations led to this finding. It is also possible that ERCC1 selectively affects micrometastasis rather than the primary tumor.

This study was unable to confirm the results of Joshi et al. (Joshi, M. B. et al. (2005) Clin. Cancer Res. 11:2215-2221) in regard to the association between TS mRNA expression and outcome for patients with esophageal cancer with adenocarcinomas and squamous cell tumors treated with trimodality therapy. Furthermore, the results of 50356 do not provide associations between outcome and polymorphisms in genes involved in DNA repair, platinum detoxification, and fluorouracil metabolism.

By treating all patients in this trial with the same regimen, it cannot be stated whether ERCC1 is truly a predictive marker to be used in selecting patients for treatment with this regimen or is simply prognostic. However, the trial validated that the predetermined cutoff level for ERCC1 has a statistically significant association with PFS and OS for patients treated with oxaliplatin, PI-FU, radiation, and surgery. Given these results, a prospective biomarkerdriven clinical trial for patients with advanced but potentially curable esophageal adenocarcinomas is under consideration.

The disclosure illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Jemal A., Siegel R., Xu J., et al.: Cancer statistics, 2010. CA Cancer J. Clin. 60:277-300, 2010.
2. Tepper J., Krasna M. J., Niedzwiecki D., et al.: Phase III trial of trimodality therapy with cisplatin, fluorouracil, radiotherapy and surgery alone for esophageal cancer: CALGB 9781. J. Clin. Oncol. 26:1086-1092, 2008.
3. Gaast A. V., van Hagen P., Hulshof M., et al.: Effect of preoperative concurrent chemoradiotherapy on survival of patients with resectable esophageal or esophagogastric junction cancer: Results from a multicenter randomized phase III study. J. Clin. Oncol. 28:302s, 2010 (suppl 15s; abstr 4004).
4. von Randen B. H., Stein H. J., Siewert J. R.: Surgical management of esophago-gastric junction tumors. World J. Gastroenterol. 12:6608-6613, 2006.
5. Suntharalingam M.: Radiation therapy for esophageal cancer. Chest Surg. Clin. N. Am. 10:569-581, 2000.
6. Meguid R. A., Hooker C. M., Taylor J. T., et al.: Recurrence after neoadjuvant chemoradiation and surgery for esophageal cancer: Does the pattern of recurrence differ for patients with complete response and those with partial or no response? J. Thorac. Cardiovasc. Surg. 138:1309-1317, 2009.
7. Ku G. Y., Ilson D. H.: Principles of system therapy, in Job B A, Thomas C. R. Jr., Hunter J. G. (eds): Esophageal Cancer: Principles of Practice. New York, N.Y., Demos Medical Publishing, 2009.
8. Vogel U., Dydahl M., Frentz G., et al.: DNA repair capacity: Inconsistency between effect of overexpression of five NER gene and the correlation to mRNA levels in the primary lymphocytes. Mutat. Res. 461:197-210, 2000.
9. Metzger R., Leichman C. G., Danenberg K. D., et al.: ERCC1 levels complement thymidylate synthase mRNA levels in predicting response and survival for gastric cancer patients receiving combination cisplatin and fluorouracil chemotherapy. J. Clin. Oncol. 16:309-316, 1998.
10. Cobo M., Is a D., Massuti B., et al.: Customizing cisplatin based on quantitative excision repair cross complementing 1 mRNA expression: A phase III trial in non-small cell lung cancer. J. Clin. Oncol. 25:2747-2754, 2007.
11. Shirota Y., Stoehlmacher J., Brabender J., et al.: ERCC1 and thymidylate synthase mRNA levels predict survival for colorectal cancer patients receiving combination oxaliplatin and fluorouracil chemotherapy. J. Clin. Oncol. 19:4298-4304, 2001.
12. Lenz H. J., Leichman C. G., Leichman L., et al.: Molecular markers as indicators of response and outcome in primary gastric cancer. Prog. Gastric Cancer Res. 2:1295-1300, 1997.
13. Khushalani N. I., Leichman C. G., Proulx G., et al.: Oxaliplatin combination with protracted infusion 5-fluorouracil and radiation: Report of a clinical trial for patients with esophageal cancer. J. Clin. Oncol. 20:2844-2850, 2002.
14. Leichman L., Lawrence D., Leichman C. G., et al.: Expression of genes related to activity of oxaliplatin and 5-fluorouracil in endoscopic biopsies of primary esophageal cancer in patients receiving oxaliplatin, 5-fluorouracil and radiation: Characterization and exploratory analysis with survival. J. Chemother. 18: 514-524, 2006.
15. Breiman L., Friedman J., Olshen R. A.: Classification and Regression Trees. Belmont, C A, Wadsworth International Group, 1984.
16. Hilsenbeck S. G., Clark G. M.: Practical p-value adjustment for optimally selected cutpoints. Stat. Med. 15:103-112, 1996.
17. Therneau T. M., Atkinson B., Ripley B.: The RPART R-Package: Recursive partitioning and regression trees. http://cran.r-project.org/web/packages/rpart/index.html.
18. Urba S. G., Orringer M. B., Turrisi A., et al.: Randomized trial of preoperative chemoradiation versus surgery alone in patients with locoregional esophageal carcinoma. J. Clin. Oncol. 19:305-313, 2001.
19. Walsh T. N., Noonan N., Hollywood D., et al.: A comparison of multimodal therapy and surgery for esophageal adenocarcinoma. N. Engl. J. Med. 335: 462-467, 1996.
20. Burmeister B. H., Smithers B. M., Gebski V., et al.: Surgery alone versus chemoradiotherapy followed by surgery for respectable cancer of the oesophagus: A randomized controlled phase III trial. Lancet Oncol. 6:659-668, 2005.
21. Stahl M., Walz M. K., Stuschke M., et al: Phase III comparison of preoperative chemotherapy compared with chemoradiotherapy in patients with locally advanced adenocarcinoma of the esophagogastric junction. J. Clin. Oncol. 27:851-856, 2009.
22. Joshi M. B., Shirota Y., Danenberg K. D., et al.: High gene expression of TS1, GSTP1 and ERCC1 are risk factors for survival in patients treated with trimodality therapy for esophageal cancer. Clin. Cancer Res. 11:2215-2221, 2005.

1. A method for aiding in the selection of or selecting or not selecting an esophageal cancer patient for a therapy comprising a platinum drug and pre-operative radiation and/or a second course of chemotherapy prior to an operative therapy, comprising determining the intratumoral expression level of an ERCC1 gene in a tumor cell or tumor tissue sample isolated from the patient, wherein the patient is selected for the therapy if the ERCC1 gene expression level is lower than a predetermined value, or the patient is not selected for the therapy if the ERCC1 gene expression level is higher than the predetermined value.

2. The method of claim 1, wherein the patient is selected for the therapy if the ERCC1 gene expression level is lower than the predetermined value.

3. The method of claim 1, wherein the patient is not selected for the therapy if the ERCC1 gene expression is higher than the predetermined value.

4. A method for aiding in the determination of or determining whether or not an esophageal cancer patient is suitable for a therapy comprising a platinum drug and pre-operative radiation and/or a second course of chemotherapy prior to an operative therapy, comprising determining the intratumoral expression level of an ERCC1 gene in a tumor cell or tumor tissue sample isolated from the patient, wherein the patient is suitable for the therapy if the ERCC1 gene expression level is lower than a predetermined value, or the patient is not suitable for the therapy if the ERCC1 gene expression level is higher than the predetermined value.

5. The method of claim 4, wherein the patient is suitable for the therapy if the ERCC1 gene expression level is lower than the predetermined value.

6. The method of claim 4, wherein the patient is not suitable for the therapy if the ERCC1 gene expression is higher than the predetermined value.

7. A method for aiding in the determination of or determining whether an esophageal cancer patient is more likely or less likely to experience progression free survival or overall survival following a therapy comprising a platinum drug and pre-operative radiation and/or a second course of chemotherapy prior to an operative therapy, comprising determining the intratumoral expression level of an ERCC1 gene in a tumor cell or tumor tissue sample isolated from the patient, wherein an ERCC1 gene expression level lower than a predetermined level determines that the patient is more likely to experience progression free survival or overall survival, or an ERCC1 gene expression level lower than the predetermined level determines that the patient is less likely to experience progress free survival or overall survival.

8. The method of claim 7, wherein an ERCC1 gene expression level lower than the predetermined level determines that the patient is more likely to experience progression free survival or overall survival

9. The method of claim 7, wherein an ERCC1 gene expression level lower than the predetermined level determines that the patient is less likely to experience progress free survival or overall survival.

10. A method for aiding in the treatment of or for treating an esophageal cancer patient selected for a therapy comprising a first or second round of an effective amount of a platinum drug prior to operative therapy, based on an intratumoral ERCC1 gene expression level in a tumor cell or a tumor tissue sample isolated from the patient that is lower than a predetermined value, comprising administering to the patient the therapy.

11. The method of claim 10, wherein the patient was selected by a method comprising determining the intratumoral expression level of the ERCC1 gene in a tumor cell or tumor tissue sample isolated from the patient.

12. A method for aiding in the treatment of or for treating an esophageal cancer patient selected for a second round of a therapy comprising administration of an effective amount of a platinum drug prior to operative therapy, based on an intratumoral ERCC1 gene expression level in a tumor cell or a tumor tissue sample isolated from the patient that is lower than a predetermined value, wherein the method comprises administering to the patient the therapy.
